(19) United States
(12) Patent Application Publication
(10) Pub. No.: US 2006/0034911 A1
(43) Pub. Date: Feb. 16, 2006

(54) New oral immediated release dosage form

(76) Inventors: Patrik Eriksson, Sodertalje (SE); Barbro Johansson, Sodertalje (SE)

Correspondence Address: COZEN O'CONNOR, P.C., 1900 MARKET STREET, PHILADELPHIA, PA, 19380, US

(21) Appl. No.: 10/537,858

(22) PCT Filed: Dec. 8, 2003
(86) PCT No.: PCT/SE03/01910

§ 371 Date: Jun. 8, 2005

(30) Foreign Application Priority Data
Dec. 9, 2002 (SE) 0203778-6

(51) Int. Cl. A61K 31/5377; A61K 9/20

(52) U.S. Cl. 424/464; 514/235.5

(57) ABSTRACT

The present invention relates to a solid oral immediate release dosage form of a pharmaceutically active compound, N-[(1,2,3,4-tetrahydro-5-methyl-8-(4-methylpiperazin-1-yl)-2-naphthyl]-4-morpholinobenzamide, in the form of the free base or pharmaceutically acceptable salts thereof. The invention further relates to processes for preparing said dosage form, the use of said dosage form and a method of prevention and/or treatment of CNS disorders and related medical disturbances using said dosage form.

### FIELD OF THE INVENTION

The present invention relates to an oral immediate release dosage form of a pharmaceutically active compound, N-[(1,2,3,4-tetrahydro-5-methyl-8-(4-methylpiperazin-1-yl)-2-naphthyl]-4-morpholinobenzamide, in the form of the free base or pharmaceutically acceptable salts thereof. The invention further relates to processes for preparing said dosage form and the use of said dosage form in therapy such as prevention and/or treatment of disorders in the CNS and related disturbances.

### BACKGROUND OF THE INVENTION

The development of a new pharmaceutically active compound is often hampered or even blocked due to unwanted physico-chemical properties of the new active compound. Some of the properties may be overcome by developing suitable pharmaceutical formulations. This is for example true for active ingredients that agglomerates upon contact with water and/or intestinal fluids and does not dissolve in a period of time that would be usable for a pharmaceutical formulation. An active compound that agglomerates upon contact with water cannot become rapidly available after administration. Such a delay in release of the active compound results in a delay of onset of action of the active compound. N-[5-methyl-8-(4-methylpiperazin-1-yl)-1,2,3,4-tetrahydro-2-naphthyl]-4-morpholinobenzamide is an active compound that agglomerates upon contact with water. N-[5-methyl-8-(4-methylpiperazin-1-yl)-1,2,3,4-tetrahydro-2-naphthyl]-4-morpholinobenzamide may be used in the prevention and/or treatment of disorders and related disturbances in the central nervous system (CNS).

Formulating N-[5-methyl-8-(4-methylpiperazin-1-yl)-1,2,3,4-tetrahydro-2-naphthyl]-4-morpholinobenzamide in a pharmaceutical composition has been difficult due to the fact that agglomerates are formed upon contact with water. The use of excipients such as binders, e.g. hydroxypropyl cellulose, microcrystalline cellulose and gelatine and the like and insoluble fillers such as microcrystalline cellulose, dibasic calcium phosphate do not prevent the active compound from forming agglomerates. The agglomerate forming properties of the active compound make it difficult to prepare an immediate release dosage form of this active compound.

It has now surprisingly been found that disintegrants, especially the so called super-disintegrants, are useful in the preparation of dosage forms with agglomerate forming active compound such as N-[5-methyl-8-(4-methylpiperazin-1-yl)-1,2,3,4-tetrahydro-2-naphthyl]4-morpholinobenzamide. It is believed that disintegrants physically prevents the primary particles of the active compound to form agglomerates in the presence of water. By using disintegrants the dosage form disintegrates in small granules upon contact with water, thereby making the active compound readily available after administration without any agglomerates being formed of the active compound.

Disintegrants are known for their wicking capacity to channel water into the interior of a pharmaceutical composition and rapidly swell in water, thereby preventing the primary particles of the active compound to form agglomerates.

Disintegrants have been used in pharmaceutical compositions like flash-melt compositions to increase the disintegration of pharmaceutical compositions. EP 1145711 describes a flash-melt composition comprising an active compound, a disintergrant, a dispersing agent, a distribution agent and a binder. This pharmaceutical composition dissolves within 25 seconds in the mouth. WO 01/76565 discloses a fast disintegration composition comprising a disintegrant, a filler, a sugar alcohol and a lubricant. This composition dissolves within 90 seconds in the mouth. WO 01/12161 discloses a process for the manufacturing of a rapid dissolving dosage form that dissolves within 30 seconds in the mouth.

The multifunctional use of disintegrants has also been described.

In WO 02/03987 disintegrants have been used to increase the stability and dissolution of poorly soluble drugs.

In WO 00/02536 describes the use of disintegrants as a disintegrant and as a taste masker. The active compound is coated with the disintegrant to cover the bitter taste of the active compound.

JP 10114655 discloses a solid preparation of an active compound that forms a gel in an acidic solution. Disintegrants are used to prevent the active compound of forming a film on the surface of the acidic solution.

The problems in obtaining a solid oral immediate release dosage form comprising an active compound such as N-[5-methyl-8-(4-methylpiperazin-1-yl)-1,2,3,4-tetrahydro-2naphthyl]-4-morpholinobenzamide, an active ingredient that agglomerates upon contact with water and/or intestinal fluids, has not been addressed well in the prior art.

There is still a need for a suitable solid oral immediate release dosage form of active compound, that forms agglomerates upon contact with water, at acid, neutral and basic pH, whereby the dosage form provides a rapid release of the active compound within a period of time that would be usable for a pharmaceutical formulation after administration in mammals.

The problem for active ingredients that agglomerates upon contact with water and/or intestinal fluids and does not dissolve in a period of time that would be usable for a pharmaceutical formulation can also be overcome by using a filler with a sufficiently high solubility in water and/or intestinal fluids.

We have now surprisingly found that disintegrants or soluble fillers may be used to prepare a solid dosage form comprising agglomerate forming active compound such as N-[5-methyl-8-(4-methylpiperazin-1-yl)-1,2,3,4-tetrahydro-2-naphthyl]-4-morpholinobenzamide. In the present invention disintegrants and/or soluble fillers physically prevents the primary particles of the active compound to form agglomerates in the presence of water and thus making it possible to have the active compound, N-[5-methyl-8-(4-methylpiperazin-1-yl)-1,2,3,4-tetrahydro-2-naphthyl]4-morpholinobenzamide, readily available after administration of the dosage form.

### DETAILED DESCRIPTION OF THE INVENTION

The present invention provides for a solid oral immediate release dosage form that is especially suitable for, in an aqueous environment, active ingredients that agglomerates upon contact with water and/or intestinal fluids and does not dissolve in a period of time that would be usable for a pharmaceutical formulation. The oral immediate release dosage form comprises an active compound, at least one disintegrant and/or at least one soluble filler, with or without one binder, and optionally other excipients, whereby the amount of the active compound may be up to 90% (w/w).

The oral dosage form of the present invention provides for a rapid release profile of the active compound in vivo having a rapid initial rise in blood plasma concentration thereby providing a fast onset of effect of the active compound. Compared to an immediate release dosage form that does not comprise a disintegrant and/or soluble filler, the present invention provides for a dosage form having less fluctuations of the intra patient-patient blood plasma concentration and thus less risk for plasma concentrations being outside the therapeutic window.

Active compounds that are specifically suitable to use in the present invention are pharmaceutically active compounds with an agglomerate-forming tendency, in an aqueous environment, at any pH.

In one aspect of the invention the oral immediate release dosage form comprises as active compound, N-[5-methyl-8-(4-methylpiperazin-1-yl)-1,2,3,4-tetrahydro-2-naphthyl]-4-morpholinobenzamide in the form of the free base or pharmaceutically acceptable salts thereof.

A further aspect of the present invention relates to the oral immediate release dosage form comprising as active compound, (R)-N-[5-methyl-8-(4-methylpiperazin-1-yl)-1,2,3,4-tetrahydro-2-naphthyl]-4-morpholinobenzamide.

Particularly suitable is the monohydrobromide salt of (R)-N-[5-methyl-8-(4-methylpiperazin-1-yl)-1,2,3,4-tetrahydro-2-naphthyl]-4-morpholinobenzamide.

(R)-N-[5-methyl-8-(4-methylpiperazin-1-yl)-1,2,3,4-tetrahydro-2-naphthyl]-4 morpholinobenzamide monohydrobromide is slightly soluble in water (6.4 mg/ml), sparingly soluble in ethanol/water 1:1 (19 mg/ml) and sparingly soluble in 0.1 M HCl (11 mg/ml). (R)-N-[5-methyl-8-(4-methylpiperazin-1-yl)-1,2,3,4-tetrahydro-2-naphthyl]4-morpholinobenzamide monohydrobromide has a plasma elimination half-life, t_{1/2}, of 35 hours in man. (R)-N-[5-methyl-8-(4-methylpiperazin-1-yl)-1,2,3,4-tetrahydro-2-naphthyl]-4-morpholinobenzamide monohydrobromide has shown to have at least five crystal modifications, named A, B, C, D and E. Any of these crystal forms A, B, C, D and E may be used in the preparation of the dosage form of the present invention. Form A is an anhydrate form and is the preferred crystal form.

The present invention relates to an oral immediate release dosage form comprising N-[5-methyl-8-(4-methylpiperazin-1-yl)-1,2,3,4-tetrahydro-2-naphthyl]4-morpholinobenzamide as the active compound, in the form of the free base or pharmaceutically acceptable salts, thereof, at least one disintegrant and/or at least one soluble filler, with or without one binder and optionally other excipients.

More specifically, the present invention relates to an oral immediated release dosage form comprising

| | | |
|---|---|---|
| N-[5-methyl-8-(4-methylpiperazin-1-yl)- | 3 to 90% | (w/w) |
| 1,2,3,4-tetrahydro-2-naphthyl]-4- | | |
| morpholinobenzamide | | |
| Disintegrants | 0 to 20% | (w/w) |
| Soluble fillers | 0 to 80% | (w/w) |
| Binders | 1 to 10% | (w/w) |
| Other excipients | up to 100% | (w/w) |

When the dosage form of the invention contains one disintegrant it may be selected from the group of carboxymethylene celluloses. For example, the disintegrant is the salt of crosslinked carboxymethylene cellulose such as a salt of an alkaline earth metal, e.g. the sodium salt.

The invention relates to the oral immediate release dosage form, whereby the disintegrants are selected from the group consisting of croscarmellose sodium, sodium starch is glycollate, crospovidone, microcrystalline cellulose, low substituted hydropropyl cellulose, soy polysaccharide, starch, alginic acid, sodium alginate, polacrillin potassium, magnesium aluminium silicate and amberlite resins.

The invention further relates to the oral immediated release dosage form wherein the disintegrant is croscarmellose sodium.

Excipients enhancing the dissolution in a neutral or acid aqueous environment, such as sodium- or potassium carbonate or -bicarbonate alone or in combination with citric acid, ascorbic acid or tartaric acid, may also be used in the oral immediate release dosage form.

The amount of disintegrants in the immediate release dosage form of the present invention may be in the range from 0 to 40% (w/w), preferably 5 to 20% (w/w).

The weight ratio of active compound to disintegrants in the oral immediate release dosage form of the present invention, may be from 6:1 to 1:2, preferably from 3:1 to 1:1.

When the dosage form of the invention contains at least one soluble filler it may be selected from the group of sugars, sugar alcohols and salts with sufficently high solubility in water at ambient conditions. Examples of water-soluble fillers are: lactose, sucrose, dextrose, mannitol, sorbitol, xylitol, maltose, maltodextrin, maltitol, lactitol, fructose, dextrates and a number of inorganic salts.

In one aspect of the invention the oral immediated release dosage form comprises binders selected from the group comprising of hydroxypropyl cellulose, microcrystalline cellulose, polyvinylpyrrolidone, gelatine, polyethylene glycol, glycerylbehenate, glycerylmonostearate, ethylcellulose, ceratonia, hydroxy propylmethylcellulose, hydroxy ethylcellulose, polydextrose, polyethyleneoxide, zein, carboxy polymethylene and carnauba wax or a mixture thereof.

A suitable binder is polyvinylpyrrolidone with an average molecular weight between 25.000 and 35.000.

The amount of binders in the immediate release dosage form of the present invention may be in the range from 0 to 20% (w/w), preferably 1 to 10% (w/w).

The weight ratio of active compound to binders may be from 8:1 to 1:2, preferably from 7:1 to 1:3.

Beside the disintegrants, soluble fillers and binders, the oral immediate release dosage form may optionally comprise other excipients, such as lubricants, fillers, flow condition agents and the like.

In one aspect of the invention the oral immediate release dosage form comprises lubricants selected from the group of magnesium stearate, calcium sterarate, zink stearate, carbomer, sodium stearyl fumarate, glyceryl monostearate, poloxamer, sodium benzoate, sodium lauryl sulphate, stearic acid, polyethylene glycol and talc.

In one aspect of the invention the oral immediate release dosage form comprises fillers selected from the group of calcium phosphates, starches, microcrystalline cellulose, calcium sulphate, polyethylene glycol, calcium carbonate, magnesium carbonate, magnesium oxide and kaolin.

In one aspect of the invention the oral immediate release dosage form comprises flow condition agents such as e.g. colloid silicon dioxide.

The amounts of these other excipients in the immediate release dosage form of the present invention may be in the range of 15 to 97% (w/w).

The dosage form may be prepared by mixing the active compound, the disintegrants, soluble fillers, binders and optionally other excipients such as lubricants, fillers and flow condition agents and the like in a suitable mixer, e.g. a Turbula mixer. The dry mix may then be filled directly into an oral dosage form.

Another route is to compress said homogeneous mixture comprising the active compound, the disintegrants, soluble fillers and the binders. These compacts may be milled through a screen and finally mixed with additional excipients such as lubricants, fillers, flow condition agents and the like and filled into an oral dosage form.

Alternatively, the dosage form may be prepared from a granulated powder. A homogeneous powder mixture may be obtained by mixing the active compound, the disintegrants, soluble fillers and optionally excipients such as binders in a suitable mixer. The mixture may then be granulated in water or another granulation liquid such as an alcohol, e.g. ethanol, methanol, isopropanol, a ketone, e.g. acetone or aqueous mixtures thereof. From an environmental point of view water is preferred. The resulting wet granules may thereafter be dried in a drying cabinet, vacuum dryer or in a fluid bed dryer and milled through a screen. The granulation may also be performed at elevated temperatures by using meltable binders. The manufactured granules may be milled through a screen. The granules are then mixed with other excipients and filled into a suitable oral dosage form.

The above processes are intended to make capsules. Other suitable oral dosage forms to be prepared by the above mentioned granules are tablets, compacted tablets, minitablets and the like.

The present invention relates to an oral immediate release dosage form, wherein the dosage form is in the form of a tablet or a capsule. The present invention also relates to processes for the manufacture of the immediate release dosage form characterized by,
Method A, comprising the steps:
Ai) mixing the active compound with the disintegrant, soluble fillers, binders and optionally lubricants, fillers and other excipients,
Aii) forming the obtained dry powder mixture into a suitable solid dosage form, Or,
Method B, comprising the steps:
Bi) mixing the active compound with the disintegrant, soluble fillers, binders and other excipients,
Bii) granulating said mixture,
Biii) optionally drying or cooling the obtained granules,
Biv) mixing the granules with other excipients,
Bv) filling the obtained dry powder mixture into a suitable solid dosage form.

Further, the present invention relates to an oral immediate release dosage form which has an in vitro dissolution profile in 50 mM acetate buffer, pH 5.5 with apparatus 2 described in USP 24, paddle method at 75 rpm, such that 85% or more of the active compound is released within 30 minutes.

The composition from which the dosage form is prepared can be formulated to contain the active compound in different amounts, e.g. between 1 and 150 mg, preferably between 5 and 120 mg, but is not limited to these intervals. These figures are presented as the free base. Suitable daily doses of the active compound may vary within a wide range and will depend on various factors such as the relevant disorder or medical conditions, the age, weight and sex, and may be determined by a physician.

The oral immediated release dosage form of the invention may thus comprise

| | | |
|---|---|---|
| N-[5-methyl-8-(4-methylpiperazin-1-yl)- | 3 to 90% | (w/w) |
| 1,2,3,4-tetrahydro-2-naphthyl]-4- | | |
| morpholinobenzamide | | |
| Disintegrants | 0 to 20% | (w/w) |
| Soluble fillers | 0 to 80% | (w/w) |
| Binders | 1 to 10% | (w/w) |
| Lubricants | 0 to 2% | (w/w) |
| Flow condition agents | 0 to 2% | (w/w) |
| Fillers | up to 100% | (w/w) |

Medical and Pharmaceutical Use

One aspect the present invention provides the use of the oral immediate release dosage form in therapy. N-[5-methyl-8-(4-methylpiperazin-1-yl)-1,2,3,4-tetrahydro-2-naphthyl]-4-morpholinobenzamide may be used as 5-HT 1B antagonists, partial agonists or full agonists, preferably as antagonists. Therefore, the oral immediate release dosage form comprising this active compound may be use in the prevention and/or treatment of disorders in the CNS and related disturbances such as 5-hydroxytrptamine mediated disorders. Examples of such disorders are disorders in the central nervous system (CNS) and related disturbances such as mood disorders (depression, major depressive disorder, major depressive episodes, dysthymia, seasonal affective disorder, depressive phases of bipolar disorder), anxiety disorders (obsessive compulsive disorder, panic disorder with/without agoraphobia, social phobia, specific phobia, generalized anxiety disorder, posttraumatic stress disorder), personality disorders (disorders of impulse control, trichotellomania), obesity, anorexia, bulimia, premenstrual syndrome, sexual disturbances, alcoholism, tobacco abuse, autism, attention deficit, hyperactivity disorder, migraine, memory disorders (age associated memory impairment, presenile and senile dementia), pathological aggression, schizophrenia, endocrine disorders (e g hyperprolactinaemia), stroke, dyskinesia, Parkinson's disease, thermoregulation, pain, hypertension. Other examples of hydroxytryptamine-mediated disorders are urinary incontinence, vasospasm and growth control of tumors (e g lung carcinoma).

Another aspect of the invention relates to the use of the oral immediate release dosage form of the present invention in prevention and/or treatment of mood disorders, anxiety disorders, personality disorders, obesity, anorexia, bulimia, premenstrual syndrome, sexual disturbances, alcoholism, tobacco abuse, autism, attention deficit, hyperactivity disorder, migraine, memory disorders, pathological aggression, schizophrenia, endocrine disorders, stroke, dyskinesia, Parkinson's disease, thermoregulatory disorders, pain, hypertension, major depressive disorder, urinary incontinence, vasospasm and growth control of tumors.

A further aspect of the present invention relates to the use of the oral immediate release dosage form of the present invetion in the manufacturing of a medicament for prevention and/or treatment of disorders in the CNS and related disturbances such as 5-hydroxytryptamine mediated disorders and any other disorders listed above.

A further aspect of the invention relates to a method for prevention and/or treatment of disorders in the CNS and related disturbances such as 5-hydroxytryptamine mediated disorders and any other disorders listed above, comprising administering to a mammal in need of such prevention and/or treatment oral immediate release dosage form of the present invention, effective for said prevention and/or treatment.

The term “rapid” as used in this specification means within 60 minutes, preferably within 30 minutes.

Abbreviations

| | |
|---|---|
| CNS | Central Nervous System |
| t | time (h) |
| t_{1/2} | plasma elimination half-life (h) |
| Cₘₐₓ | Maximum plasma drug concentration (nmol/L) |
| tₘₐₓ | Time to reach maximum plasma drug concentration |
| | following drug administration (h) |
| PEG | Polyethylene glycol |
| PVP | Polyvinylpyrrolidone |

### EXAMPLES

The invention will now be illustrated by the following non-limiting example.

### Example 1

The following components, expressed as mg per capsule, were used in order to manufacture 50 mg capsules; batch size 28000 capsules:

| | |
|---|---|
| Active compound: | 59 |
| PVP K-25 | 8.9 |
| Croscarmellose sodium | 17.9 |
| Mannitol | 93 |
| Water | 71.5 |
| Magnesium stearate | 0.45 |
| Colloidal silicon dioxide | 0.45 |

The active compound, (R)-N-[5-methyl-8-(4-methylpiperazin-1-yl)-1,2,3,4-tetrahydro-2-naphthyl]-4-morpholinobenzamide monohydrobromide, were screened through a 0.5 mm square screen. PVP K-25 and croscarmellose sodium were added and all the ingredients were thereafter mixed in a Turbula mixer for 10 minutes at 30 rpm.

The powder mixture was then transferred to a high shear mixer. Mannitol, sieved though a 0.5 mm square screen, was added and the powder mass was further mixed for 10 minutes at 150 rpm. This powder mixture was then granulated with water in the high shear mixer for 2 minutes and 45 seconds at 150 rpm. A chopper was used during the last 15 s at 2000 rpm. The formed wet granules were dried in a drying cabinet at +50° C. for 5 hours. The granules were milled in an oscillating granulator through a screen of 1.00 mm. The dry granules were then mixed with colloidal silicon dioxide (screened through 0.5 mm) in a Turbula mixer for 3 minutes at 30 rpm. Magnesium stearate was added through a screen of 0.5 mm and the mixing was continued for further 45 seconds.

The final homogeneous dry powder mixture was filled into hard gelatine capsules size no. 1, colour Swedish orange, in a capsule-filling machine.

In order to test the release rate of the active drug compound from the capsules an in vitro dissolution of the capsule was accomplished by using the USP paddle method, 75 rpm.
(Dissolution Test, USP 24)
Used conditions:
Medium: acetate buffer, pH=5.5, volume: 1000 ml, temperature: +37° C.

The following results were obtained:

| Time | Amount |
|---|---|
| (min) | dissolved % |
| 0 | 0 |
| 5 | 5 |
| 10 | 26 |
| 15 | 48 |
| 20 | 68 |
| 25 | 86 |
| 30 | 98 |
| 45 | 101 |
| 60 | 102 |

### CONCLUSION

From the Example it is evident that with the oral dosage form according to the present invention an immediate release is achieved by using the disintegrant.

Bioavailability

A single dose bioavailability study was performed in healthy volunteers. Two different formulations of (R)-N-[5-methyl-8-(4-methylpiperazin-1-yl)-1,2,3,4-tetrahydro-2-naphthyl]-4-morpholinobenzamide monohydrobromide were tested. One group of fasting 6 volunteers received (R)-N-[5-methyl-8-(4-methylpiperazin-1-yl)-1,2,3,4-tetrahydro-2-naphthyl]4morpholinobenzamide monohydrobromide as an aqueous solution (n=6). The other group of 5 volunteers received (R)-N-[5-methyl-8-(4-methylpiperazin-1-yl-1,2,3,4-tetrahydro-2-naphthyl]+morpholinobenzamide monohydrobromide in an immediate release capsule (n=5). The composition of the capsule is according to example 1, except that the concentration of the active compound was lower (3.3%). The dose in both dosing groups was 15 mg (calculated as the base). Plasma samples were withdrawn prior to and up to 200 hours after drug administration (for solution dosing group up to 48 hours). Determination of (R)-N-[5-methyl-8-(4-methylpiperazin-1-yl)-1,2,3,4-tetrahydro-2-naphthyl]-4-morpholinobenzamide monohydrobromide in the plasma was performed using liquid chromatography-tandem mass spectrometry (LC-MS-MS). After oral administration of (R)-N-[5-methyl-8-(4-methylpiperazin-1-yl)-1,2,3,4-tetrahydro-2-naphthyl]4-morpholinobenzamide monohydrobromide, the following pharmacokinetic parameters of the corresponding base were estimated: maximum plasma drug concentration (Cₘₐₓ), time to reach Cₘₐₓ following drug administration (tₘₐₓ) area under plasma concentration-time curve from zero to infinity (AUC_{(0-∞)}), terminal half-life (t_{1/2}) and oral clearance (CL/F). The results are presented in Table A below.

TABLE A
Pharmacokinetic data obtained after administration of an oral solution compared to
the oral immediate release dosage form of Example 1. Dose 15 mg (as the base)

| | | | Cₘₐₓ | t_{1/2} | AUC_{(0-∞)} | CL/F |
|---|---|---|---|---|---|---|
| Dosing form | | tₘₐₓ (hours) | (nmol/L) | (hours) | (nmol * h/L) | (L/h) |
| Oral | Mean | 5.0 | 31.7 | 35.2 | 1314 | 26.5 |
| solution | SD | 2.2 | 13.4 | 8.0 | 290 | 6.1 |
| (n = 6) | Median | 5.5 | 26.0 | 33.7 | 1295 | 25.9 |
| | Range | 2.0-8.0 | 20.3-49.1 | 25.7-47.6 | 918-1744 | 19.2-36.4 |
| Capsules | Mean | 4.6 | 32.9 | 37.1 | 1295 | 26.2 |
| 3 × 5 mg | SD | 1.7 | 8.2 | 6.2 | 176 | 3.6 |
| (n = 5) | Median | 5.0 | 35.7 | 35.4 | 1254 | 26.7 |
| | Range | 3.0-7.0 | 23.0-41.0 | 30.2-44.1 | 1084-1479 | 22.6-30.8 |

The results show that the oral immediate release dosage form according to the present invention provides a blood plasma profile of the active compound similar to when the active compound is administered orally in an aqueous solution. This is valid for both the tₘₐₓ and the Cₘₐₓ. The initial rise in blood plasma concentration is achieved by administration of the active compound in the oral immediate release dosage form of the present invention.

1-29. (canceled)

30. An oral immediate release dosage form comprising N-[5-methyl-8-(4-methylpiperazin-1-yl)-1,2,3,4-tetrahydro-2-naphthyl]-4-morpholinobenzamide as the active compound, in the form of the free base or pharmaceutically acceptable salt, thereof, at least one disintegrant and/or at least one soluble filler, with or without one binder, and optionally other excipient.

31. The oral immediated release dosage form according to claim 30 wherein the active compound is (R)-N-[5-methyl-8-(4-methylpiperazin-1-yl)-1,2,3,4-tetrahydro-2-naphthyl]-4-morpholinobenzamide.

32. The oral immediated release dosage form according to claim 30 wherein the salt of (R)-N-[5-methyl-8-(4-methylpiperazin-1-yl)-1,2,3,4-tetrahydro-2-naphthyl]-4-morpholinobenzamide is (R)-N-[5-methyl-8-(4-methylpiperazin-1-yl)-1,2,3,4-tetrahydro-2-naphthyl]-4-morpholinobenzamide monohydrobromide.

33. The oral immediate release dosage form according to claim 30 wherein the disintegrant is selected from the group consisting of croscarmellose sodium, sodium starch glycollate, crospovidone, microcrystalline cellulose, low substituted hydropropyl cellulose, soy polysaccharide, starch, alginic acid, sodium alginate, polacrillin potassium, magnesium aluminium silicate, and amberlite resin.

34. The oral immediate release dosage form according to claim 33 wherein the disintegrant is croscarmellose sodium.

35. The oral immediate release dosage form according to claim 30 wherein the soluble filler is selected from the group consisting of lactose, sucrose, dextrose, mannitol, sorbitol, xylitol, maltose, maltodextrin, maltitol, lactitol, fructose, dextrate, and an inorganic salt.

36. The oral immediate release dosage form according to claim 30 wherein the soluble filler is mannitol.

37. The oral immediate release dosage form according to claim 30 wherein the binder is selected from the group consisting of hydroxypropyl cellulose, microcrystalline cellulose, polyvinylpyrrolidone, gelatine, polyethylene glycol, glycerylbehenate, glycerylmonostearate, ethylcellulose, ceratonia, hydroxy propylmethylcellulose, hydroxy ethylcellulose, polydextrose, polyethyleneoxide, zein, carboxy polymethylene, and carnauba wax, or a mixture thereof.

38. The oral immediate release dosage form according to claim 37 wherein the binder is polyvinylpyrrolidone.

39. The oral immediate release dosage form according to claim 30 wherein the other excipient is a lubricant, filler, or flow condition agent.

40. The oral immediate release dosage form according to claim 39 wherein the lubricant is selected from the group consisting of magnesium stearate, calcium sterarate, zink stearate, carbomer, sodium stearyl fumarate, glyceryl monostearate, poloxamer, sodium benzoate, sodium lauryl sulphate, stearic acid, polyethylene glycol, and talc.

41. The oral immediated release dosage form according to claim 39 wherein the filler is selected from the group consisting of calcium phosphate, starch, microcrystalline cellulose, calcium sulphate, polyethylene glycol, calcium carbonate, magnesium carbonate, magnesium oxide, and kaolin.

42. The oral immediate release dosage form according to claim 30 wherein the other excipient is sodium- or potassium carbonate or -bicarbonate alone or in combination with citric acid, ascorbic acid, or tartaric acid.

43. The oral immediate release dosage form according to claim 39 wherein the flow condition agent is colloid silicon dioxide.

44. The oral immediate release dosage form according to claim 30 wherein the ratio of active compound to disintegrant is from 6:1 to 1:2.

45. The oral immediate release dosage form according to claim 30 wherein the ratio of active compound to disintegrant is from 3:1 to 1:1.

46. The oral immediate release dosage form according to claim 30 wherein the weight ratio of active compound to binder is from 8:1 to 1:2.

47. The oral immediate release dosage form according to claim 30 wherein the dosage form is in the form of a capsule or a tablet.

48. The oral immediate release dosage form according to claim 30 whereby the dosage form has a mean dissolution profile in vitro, in 50 nM acetate buffer, pH of 5.5, using USP Paddle method at 75 rpm, such that at least 85% of the active compound is released within 30 minutes.

49. An oral immediated release dosage form comprising 3 to 90% (w/w) N-[5-methyl-8-(4-methylpiperazin-1-yl)-1,2,3,4-tetrahydro-2-naphthyl]-4-morpholinobenzamide, 0 to 20% (w/w) disintegrant, 0 to 80% (w/w) soluble filler, 1 to 10% (w/w) binder, and up to 100% (w/w) other excipient.

50. A method for the manufacture of an oral immediate release dosage form according to claim 30 comprising:
Method A, comprising the steps:
Ai) mixing the active compound with the disintegrant, soluble filler, binder, and optionally lubricant, filler and other excipient; and
Aii) forming the obtained dry powder mixture into a suitable solid dosage form; or
Method B, comprising the steps:
Bi) mixing the active compound with the disintegrant, soluble filler, and optionally binder and other excipient;
Bii) granulating said mixture;
Biii) optionally drying or cooling the obtained granules;
Biv) mixing the granules with other excipient; and
Bv) filling the obtained dry powder mixture into suitable solid dosage form.

51. A method of preventing and/or treating a disorder in the central nervous system of a mammal comprising contacting a mammal with an oral immediate release dosage form according to claim 30.

52. The method of claim 51 wherein the disorder is a mood disorder, anxiety disorder, personality disorder, obesity, anorexia, bulimia, premenstrual syndrome, sexual disturbance, alcoholism, tobacco abuse, autism, attention deficit, hyperactivity disorder, migraine, memory disorder, pathological aggression, schizophrenia, endocrine disorder, stroke, dyskinesia, Parkinson's disease, thermoregulatory disorder, pain, or hypertension.

53. The method of claim 51 wherein the disorder is major depressive disorder.

54. The method of claim 51 wherein the disorder is urinary incontinence, vasospasm, or growth control of a tumor.

55. The method of claim 51 wherein the disorder is a 5-hydroxytryptamine mediated disorder.

56. The oral immediate release dosage form according to claim 30 whereby the dosage form upon administration provides tₘₐₓ for (R)-N-[5-methyl-8-(4-methylpiperazin-1-yl)-1,2,3,4-tetrahydro-2-naphthyl]-4-morpholinobenzamide monohydrobromide between 3 to 7 hours.

57. A method of preparing an oral immediate release dosage form of an active compound that forms an agglomerate upon contact with water, at acidic, neutral or basic pH comprising formulating the active compound with a disintegrant.
